(19) United States
(12) Patent Application Publication
(10) Pub. No.: US 2025/0339021 A1
(43) Pub. Date: Nov. 6, 2025

(54) SOFT TISSUE RETRACTORS

(71) Applicant: WRMDSV IV, LLC, Charleston, SC (US)

(72) Inventors: Walter G. Renne, Charleston, SC (US); Michael DeFee, Charleston, SC (US)

(21) Appl. No.: 18/656,545

(22) Filed: May 6, 2024

(51) Int. Cl. A61B 1/24

(52) U.S. Cl. CPC A61B 1/24

(57) ABSTRACT

An expandable soft tissue retractors comprising a controlling handle portion and a soft tissue retracting portion, wherein the controlling handle portion may include a biasing element to facilitate angular pivoting of the soft tissue retracting portion when the controlling handle portion is manipulated is disclosed. The soft tissue retractors are sufficiently small in their normal (or non-use), substantially constricted or contracted state so as to be capable of being inserted into a patient's soft tissue area (e.g., the mouth) without appreciable discomfort, and then expanded to retract tissue once comfortably placed in their proper locations. The retractors are not only easy to control and manipulate, but also feature an anatomical design that optimizes soft tissue retraction for the maxilla and mandible.

### FIELD OF THE INVENTION

The present invention relates generally to soft tissue retraction devices. More particularly, the present invention relates to soft tissue retractors that may be used in dental or medical applications.

### BACKGROUND OF THE INVENTION

Soft tissue retraction is an essential component activity in the production of intraoral digital impressions. For example, by retracting the tissues of a patient's oral cavity, a clinician can better see and, therefore, properly scan the area of interest using optical scanners such as an intraoral scanner to capture the hard and soft tissues of the mouth. However, optically-scanned impressions are sensitive to tissue movement, and, in the preparation of such impressions, soft tissue often gets in the way of the scanner, thereby causing inaccurate scans.

Many traditional retractors currently on the market are only available in fixed sizes that are usually designated as small, medium, and large. Thus, in practice, a dentist may be required to stock multiple different sizes of retractors and yet—at the same time—have only a limited number of fixed available options when attempting to select the proper size needed for a given patient and procedure. Often, a retractor with the wrong size is selected, such as one too large, which can be painful for the patient when the retractor stretches the tissue too much. Additionally, there is no customization or adjustability to the size of the individual retractor, often making it difficult to get optimal retraction. Nevertheless, retractors that are larger than the mouth opening are often needed, and, as discussed above, this can cause painful stretching of the lips during fitting and insertion of the retractors into the mouth. Despite the need for retractors that are larger than the mouth, smaller than optimally-sized retractors are often selected to minimize or eliminate painful stretching. However, this practice of selecting smaller than optimally-sized retractors results in improper retraction, and, consequently, a poor digital impression.

Various types of retractors have been proposed by others. But on information and belief, none address the above-described problem in the same way as in the present disclosure, as explained in the description further below.

For example, CN217162178U (Jiang et al.), published Aug. 12, 2022, discloses a soft tissue draw hook for maxillofacial oral surgery. The soft tissue draw hook is described as comprising a handle, a ring part that is fixedly connected with the handle, a first hook strip, and a second hook strip, wherein the first hook strip and the second hook strip are connected to the ring part. The reference states that the first hook strip is fixedly connected to the ring part, and the second hook strip is movably connected to the ring part. The reference further teaches that the ring part has a circular ring shape, the first hook strip and the second hook strip are both arranged on an outer wall of the ring part, the second hook strip rotates along the outer wall of the ring part, and an angle is formed between the second hook strip and the first hook strip, so that two parts of oral soft tissue are pulled at the same time. According to the reference, the soft tissue retractor for the maxillofacial surgical operation can be used for quickly shaping and adjusting the size of an exposed surgical field, is simple in structure and convenient to operate, facilitates exposure of the surgical field in the operation, and saves manpower.

CN114041968A (Zhang et al.), published Feb. 15, 2022, discloses a mouth gag comprising a first grab handle, the middle of the first grab handle being movably connected to a connecting shaft, the lower end of the first grab handle being provided with a second grab handle, the left end of the first grab handle being movably connected to a first supporting arm, the left end of the first supporting arm being movably connected to a second supporting arm, and an adjusting mechanism being arranged on the outer surface of the first supporting arm. The reference states that when the occlusal force between an upper pressure plate and a lower pressure plate exceeds a set value, under the interaction force of a rack and a clamping block, the clamping block is forced by the rack to move rightwards so that the rack slides downwards, and when the rack slides to be staggered by one tooth, the force borne by the upper pressure plate due to the lower pressure plate is reduced, and thus the rack and the clamping block are kept engaged. The reference states that when the critical value of rehabilitation occlusal force is reached, the rehabilitation mouth gag is automatically relaxed, and strain is prevented.

CN109172009B (Sun et al.), published Mar. 9, 2021, discloses a medical device for oral examination that is said to provide a better medical experience for a patient, comprising: a scissor pair comprising a first scissor arm and a second scissor arm, wherein the two scissor arms are crossed with each other and are rotatably connected to each other through a shared rotating shaft; a first tooth support sleeve rotatably connected to a first end of the first scissor arm for supporting an upper row of teeth of a patient; a second tooth support sleeve screwed to the first end of the second scissor arm for supporting the lower row of teeth of the patient; a tongue depressor arranged on the end head of the first end of the second scissor arm; a sliding cylinder with two ends respectively connected to second ends of the two scissor arms; an actuating lever, a first end of the actuating lever being rotatably connected to the first scissor arm and a second end of the actuating lever being slidably connected to the second scissor arm; and a pull rod, wherein the first end of the pull rod is rotatably connected to the execution rod, and the second end of the pull rod is slidably supported on the second scissor arm. The instrument is disclosed as being convenient for controlling the opening amplitude and the force of the oral cavity, as having better applicability, and as ensuring that the patient experiences better medical experience.

IN202011043418A (All India Institute of Medical Sciences), published Oct. 7, 2022, discloses an intra-oral reduction and fixation system comprising unique forceps with special elements that include an arrestable clamp that aids in holding a condyle, a plate carrier system that aids in positioning the plate, and a reduction system that helps in maintaining the position of reduced condyle. According to the reference, an endoscopic channel that is provided along the shaft of the device can be utilized for inserting a telescopic device or endoscope, wherein a tissue retractor associated with the reduction plate carrier system is present to facilitate better visibility and access to the fractured site of mandibular condyle.

U.S. Pat. No. 10,772,493B2 (Choi et al.), issued Sep. 15, 2020, discloses that, to accurately capture the image of an inside of an oral cavity, a dental retractor is provided including a retracting base portion that includes an inner retracting portion extending in an arch-shaped profile to surround an inner portion of gum at any one side of an upper jaw and a lower jaw inside an oral cavity and having a pressing holding portion formed at an outer peripheral surface so that a tongue is accommodated therein and an outer retracting portion integrally extending from both ends of the inner retracting portion and extending in an arch-shaped profile to surround an outer portion of the gum, and a handle portion including a grip portion connected outward from the retracting base portion.

WO0150973A1 (Lee et al.), published Jul. 19, 2001, discloses a mandibular retractor consisting of a handle (1), and its frame (2), which is bent 45 degrees from the handle and provided with a hook-shaped tip loop (2a), a space on loop (2b), and an end (3a) of an inserted tip (3) in the frame, and axis (4a) and lever (4), which is squeezed to move the inserted tip part.

CN107811713A (Lin et al.), published Mar. 20, 2018, discloses a metal post pin removing device comprising a clamping frame I, wherein a rotating slot I is formed in an upper surface of the clamping frame I, the upper surface of the rotating slot I is rotatably connected to a stationary shaft, the clamping frame I is rotatably connected to a clamping frame II by virtue of the stationary shaft, a rotating slot II is formed at a position, close to the stationary shaft, of the lower surface of the clamping frame II, a mounting plate is arranged on the upper surface of the stationary shaft, and a current condenser is arranged on the upper surface of the mounting plate. The reference states that metal post pin can be clamped effectively by virtue of a clamping plate I, a screw thread I, a screw thread II, and a clamping plate II, and, therefore, an oral cavity of a patient can be illuminated conveniently by virtue of an LED illuminating lamp, so that a doctor can operate conveniently. The reference states that the clamping frame I and the clamping frame II can keep a clamping state by virtue of a spring, so that use and placement are facilitated; and the metal post pin removing device can be conveniently hung by virtue of a hanging ring, so that the metal post pin removing device is easy to use.

CN111568356A (Sun et al.), published Aug. 25, 2020, discloses a mouth gag, the main body of which is a pair of handles that are opened in a forceps shape under its natural condition; the right ends of the handles are hinged through a hinged screw; the pair of handles and the hinged screw form lever shapes, which are mutually supported; the handles extend out long arm ends towards the right edges through the hinged screw; isosceles triangular opening tips are arranged at the tail ends of long arms; a wall support is arranged between the two handles; the wall support is a folded angle-shaped metal sheet; a cylindrical blocking boss is arranged on the wall support; the wall support is made of spring steel; and sawtooth-shaped blocking pieces are arranged on the surfaces of the two handles. The reference states that when a patient suddenly suffers from sudden cardiac arrest and is subjected to emergency cardiopulmonary resuscitation rescue, the oral cavity can be quickly opened by using the first-aid oral mouth gag to remove oral secretions or foreign matter so as to unblock the airway, and the success rate of cardiopulmonary resuscitation rescue is greatly improved by using an oropharyngeal airway or a trachea cannula.

An oral expander that is sterilizable, that may be autoclaved (<130° C.), and can be used as a cheek retractor/mouth opener/lip retractor/cheek expander is available at https://stardent2008.en.made-in-china.com/product/ROfAslyCEGVW/China-Dental-Mouth-Opener-Lip-Retractor-Cheek-Expander-Stainless-Steel-with-Rubber-on-Tip-Material-Dentist-Tools-Large-Small-Size.html.

### SUMMARY OF THE INVENTION

Unlike the aforementioned devices proposed by others, the present invention solves the problem identified above by providing expandable soft tissue retractors that are sufficiently small in their normal (or non-use), substantially constricted or contracted state so as to be capable of being inserted into a patient's soft tissue area (e.g., the mouth) without appreciable discomfort, and then expanded to retract tissue once comfortably placed in their proper locations. The retractors are not only easy to control and manipulate, but also feature an anatomical design that optimizes soft tissue retraction for the maxilla and mandible. Their ease of control and manipulation as well as anatomical shapes, combined with their ability to expand and contract to properly fit the unique patient, make the devices according to the present invention particularly suitable for achieving painless and optimal retraction, thereby satisfying a long-felt need in the art and solving the problem identified above.

The present invention, described below by way of non-limiting aspects, embodiments, or examples, is directed to a retractor comprising a controlling handle portion and a soft tissue retracting portion. The controlling handle portion may include a biasing element, a first elongated gripping member, and a second elongated gripping member, wherein each elongated gripping member extends from or is attached to the biasing element. The biasing element may be configured to allow the elongated gripping members to pivot angularly or separate from each other by a predetermined distance when the elongated gripping members are manually urged towards each other by pressing or squeezing (e.g., with fingers or by mechanical means) and to revert back to their original positions when the pressing or squeezing is relaxed or removed. The soft tissue retracting portion may include a first arm and a second arm, with each arm having a proximal section that extends from or is attached to one of the elongated gripping members of the controlling handle portion and a distal section that is configured to contact and retract tissue. Hence, the convenient and easy manipulation of the elongated gripping members simultaneously controls the contraction or expansion (by angular displacement) of the distal sections of the arms relative to each other. The elongated gripping members of the controlling handle portion and the arms of the tissue retracting portion may be configured such that the distal sections of each of the arms are substantially contracted relative to each other in the normal bias position of the biasing element, thus allowing the distal sections of the first and second arms to be easily insertable into a vestibule of the mouth, but can be expanded as desired to retract soft tissue when the elongated gripping members of the controlling handle portion are urged towards each other by pressing or squeezing. The soft tissue retracting portion can be shaped and sized to conform anatomically.

In one aspect of the invention, the biasing element may have a curved shape.

In another aspect, the biasing element may be generally arch-shaped or generally C-shaped.

In another aspect, the biasing element is contained within a housing.

In another aspect, the generally arch-shaped or generally C-shaped biasing element is a spring-loaded mechanism.

In another aspect, the arms of the tissue retracting portion are configured to intersect in an overlaid, scissor configuration in both their substantially contracted and expanded states.

In another aspect, the arms of the tissue retracting portion are configured to intersect in an overlaid, scissor configuration in the substantially contracted and/or expanded states.

In a further aspect of the invention, the arms of the soft tissue retracting portion may be configured to intersect in an overlaid, scissor configuration in the substantially contracted state.

In another aspect, the retractor may be configured as a mandibular retractor.

In another aspect, the retractor may be configured as a maxillary retractor.

In yet another aspect, the retractor may be constructed from metallic materials, polymeric materials (e.g., polylactic acid (PLA)), fiber-reinforced materials (carbon fiber materials), and combinations thereof.

In yet another embodiment, a method of using the retractor to retract soft tissue, especially in the context of producing optically-scanned impressions is contemplated.

Other more specific exemplary embodiments are described in further detail below.

### BRIEF DESCRIPTION OF THE DRAWINGS

The preferred embodiments of the invention will hereinafter be described in conjunction with the appended drawings provided to illustrate but not limit the invention, where any like designations denote like elements, and in which:

FIG. 1 (prior art) illustrates a common but inconvenient practice of using two conventional devices such as a dental mouth mirror and dental cotton pliers, usually manipulated by two persons, to retract soft tissues in an oral cavity;

FIG. 2 shows an exemplary perspective view of a retractor according to an embodiment of the present disclosure;

FIG. 3 shows another exemplary perspective view of the retractor shown in FIG. 2;

FIG. 4 shows another exemplary perspective view from the other side of the retractor shown in FIG. 3;

FIG. 5 shows the expansion (angular displacement) of the distal portions of the arms of the retractor shown in FIGS. 2-4;

FIG. 6 illustrates an exemplary insertion of the retractor shown in FIGS. 2-5 into an oral cavity to separate the soft tissues around a contoured portion of the lower gums;

FIG. 7 shows the top views of an exemplary alternative embodiment of the retractor in the form of a maxillary retractor at various states as the elongated gripping members of the handle portion are manipulated to thereby expand the distal portions of the arms;

FIG. 8 shows an exemplary top view of the maxillary retractor according to FIG. 7, illustrating the angular displacement of the distal portions of the arms in a substantially expanded state; and

FIG. 9 illustrates an exemplary insertion and placement of the maxillary retractor of FIGS. 7 and 8 into an oral cavity around the lower gums.

Like reference numerals refer to like parts throughout the several sheets of the drawings.

### DETAILED DESCRIPTION OF THE INVENTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims.

Some features may be described using positional, sequential, or relative terms, such as “first,” “second,” “proximal,” or “distal.” Such terms are for reference with respect to the appended Figures and are not meant to limit the disclosed embodiments or to exclude additional components, unless explicitly limited as such in the appended claims.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific amounts, dimensions, materials, and/or other physical characteristics relating to the embodiments disclosed herein are not to be considered as limiting but merely exemplary, unless the claims expressly state otherwise.

Furthermore, a person skilled in the art would understand from reading this disclosure that “a” and “an” each generally denotes “at least one,” but does not exclude a plurality unless the contextual use dictates otherwise. Also, the term “or” in a list of enumerated items denotes “at least one of the items” and, therefore, does not exclude a plurality of items of the list.

As described above, the idea underling the present invention pertains to providing a soft tissue retractor that is capable of being inserted into and placed in a patient's soft tissue area of interest in a compact, contracted state such that the patient experiences minimal or no discomfort and thereafter manipulated to an expanded state to cause retraction of the surrounding soft tissues such that the area of interest can be better viewed (e.g., for examination of an oral cavity).

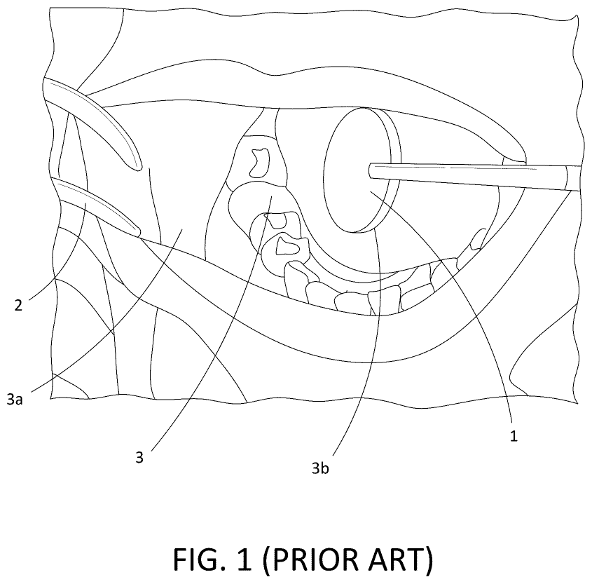

The present invention provides a significant advantage over the prior art, such as that illustrated in FIG. 1 (prior art), which shows a typical situation where two people and two tools are needed to perform the retraction. For example, one person holds a tool such as a dental cotton plier 2 to move away cheek soft tissue 3a in a patient's oral cavity 3, while another person holds a second tool such as a dental mirror 1 to move away the patient's tongue 3b. As may be readily apparent, this practice is labor intensive, may be painful to the patient, and may not be convenient or easy in providing a clear unimpeded view of the area of interest for a prolonged period of time.

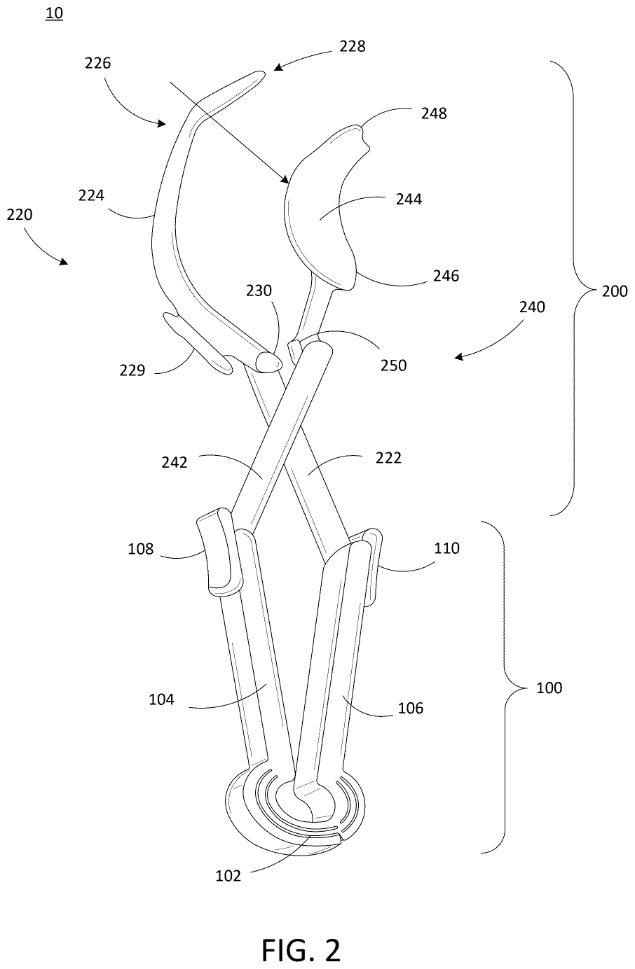

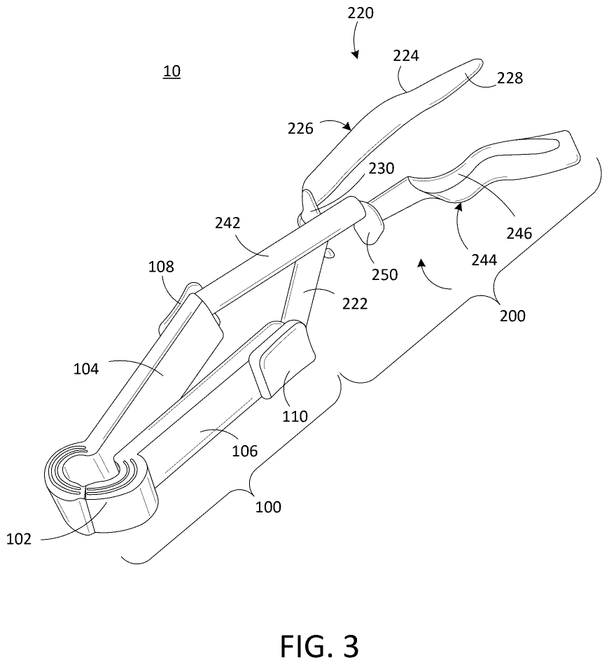

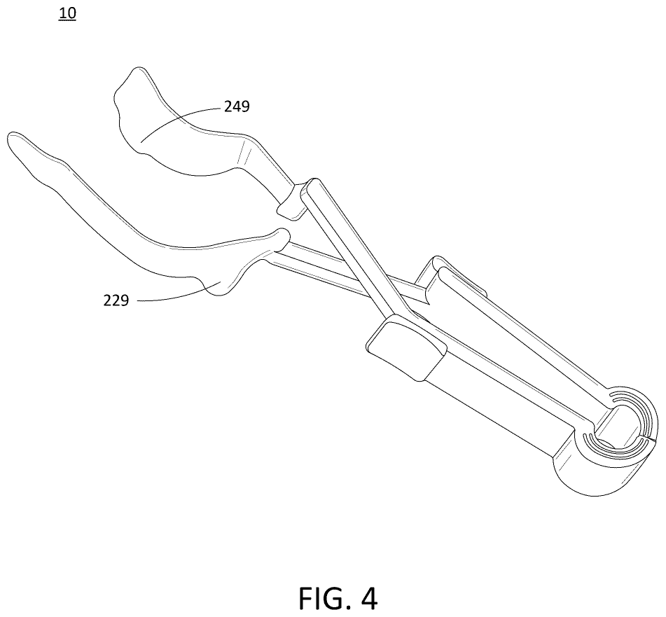

By contrast, in one embodiment of the present invention with reference to FIGS. 2-6, a retractor 10, such as a mandibular retractor 12 or maxillary retractor 30, may be easily inserted into a patient's oral cavity 3 with one hand, causing minimal or no discomfort to the patient, and thereafter manipulated to an expanded state by pressing or squeezing elongated gripping members 104 and 106 such that proper placement and optimized soft tissue retraction can be facilitated. That is, retractor 10, such as mandibular retractor 12 or maxillary retractor 30, can be comfortably placed in the patient's mouth in a substantially contracted or contracted position and then expanded to retract both the cheek and tongue at the same time.

Specifically, as shown in FIG. 2, the retractor 10 such as a mandibular retractor 12, according to an embodiment of the present invention may comprise a controlling handle portion 100 and a tissue retracting portion 200. The retractor 10 may be in the form of a single unitary or monolithic structure or may comprise multiple attached (i.e., connected, fastened, fused, or bonded) components that may be assembled together according to conventional techniques.

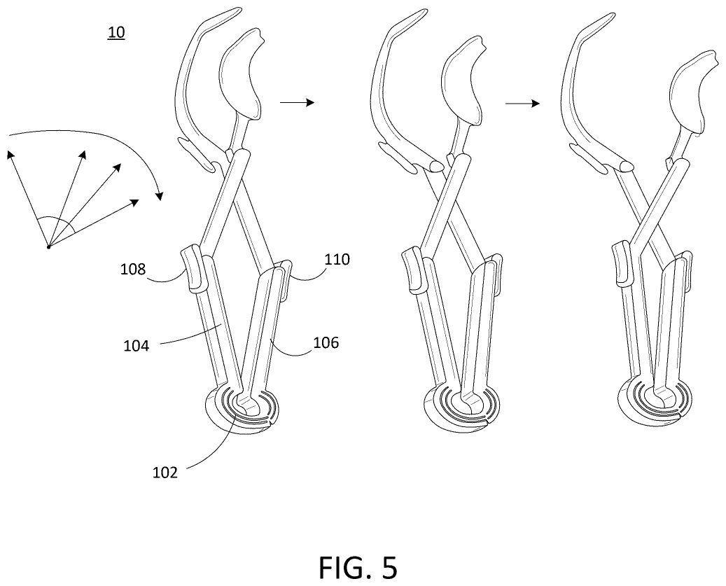

The controlling handle portion 100 may include, for example, a generally arch- or generally C-shaped biasing element 102 provided with, for example, a biasing mechanism such as a spring (not shown) in a housing and two elongated gripping members 104 and 106 that extend from or is attached to the biasing element 102. It is within the scope of the present invention to implement mechanisms other than a spring, such as, but not limited to, metallic or polymeric materials that are somewhat flexible yet can revert to its original shape, thereby providing an inherent biasing force. The biasing element 102 may be configured to ensure that the distal sections of the tissue retracting portion 200 are substantially constricted or contracted when the retractor 10 is not in use and expanded when the controlling handle portion 100 is manipulated (such as by exerting a pressing or squeezing force on finger pads or rests 108 and 110, which may be provided on the elongated gripping members 104 and 106, respectively, for ergonomic operation) after entry into the patient's oral cavity 3, as illustrated in FIG. 5.

The ergonomic design of finger pads or rests 108 and 110 provide user comfort and ease of manipulation. As mentioned previously, it should be appreciated by one skilled in the art that any of the parts of the retractor 10, such as the elongated gripping members 104 and 106, may be unitary or monolithic with respect to one or more other parts of the retractor 10, such as the finger rests 108 and 110, respectively.

The biasing element 102 exemplified in FIGS. 2-6 is in the form of a spring mechanism that may comprise, e.g., one or more curved or generally omega-shaped, open-ended springs (e.g., metallic) that are available in commerce. However, it is possible to implement other spring mechanisms (e.g., coiled springs) as long as the mechanisms can perform the described function.

The soft tissue retracting portion 200 includes two arms 220 and 240 including: elongated extension members 222 and 242, respectively, that are arranged to intersect in an overlaid, scissor configuration relative to each other and are proximal to the controlling handle portion 100; and soft tissue retracting members 224 and 244, respectively, that are distal to the controlling handle portion 100. The elongated extension members 222 and 242 or the soft tissue retracting members 224 and 244 may be provided with protrusions or tabs 230 and 250, respectively, to act as stops in the non-use position (i.e., to counteract the biasing force exerted by the biasing element). It should also be understood by one skilled in the art that the elongated extension members 222 and 242 are designed to provide adequate placement of the retractor 10 into the vestibule of the mouth. Again, it should be appreciated by one skilled in the art that any part(s) of the soft tissue retracting portion 200 and any part(s) of the controlling handle portion 100 may be manufactured as a unitary or monolithic structure as illustrated in the Drawings.

In the embodiment shown in FIGS. 2-6, the arms 220 and 240 may be shaped and sized differently. Specifically, soft tissue retracting member 224 may be longer than soft tissue retracting member 244 and may be provided with different features for manipulating different anatomical features. Soft tissue retracting member 224 may be configured to include a buccal shelf contour 226, a retromolar pad contour 228, and a labial vestibule extension 229 as shown in FIGS. 2-4. On the other hand, soft tissue retracting member 244 may be configured to include a glossal hood 246, a retromylohyoid fossas extension 248, and a lingual vestibule extension 249.

Optionally, the elongated extension members 222 and 242 of the arms 220 and 240, respectively, may be provided with, but not limited to, a slide system or ratchet system (not shown) for expansion and contraction.

The slide system may comprise, e.g., a fixed pin/tab on one of the extension members 222 and 242 that can slide within a corresponding groove on the other of the extension member 222 and 242, whereby the pin/tab can be locked in a desired position to provide a desired expansion or contraction.

The ratchet system may comprise, e.g., notches/recesses (not shown) in the form of a serrated teeth structure along a length of one of the extension members 222 and 242 and corresponding protrusions/recesses on the other of the extension members 222 and 242.

The retractor 10 may be a mandibular retractor 12 and made from various suitable materials including, but not limited to, one or more of metallic materials, polymeric materials (e.g., PLA), reinforced fiber materials (carbon fiber-containing materials), composites, and combinations thereof. The materials of construction for retractor 10, such as the mandibular retractor 12 or maxillary retractor 30, may be selected to form a device that is either disposable (in whole or part, such as the soft retracting members) or completely reusable (with autoclaving capability).

Referring to FIG. 5, it can be seen that as the elongated gripping members 104 and 106 are manipulated (i.e., pressed or squeezed towards each other such as with finger forces), the soft tissue retracting members 224 and 244 are urged to expand relative to each other by a certain angular displacement (represented by the dotted arrows). In this manner, an expandable angle created by pressing the retractor 10 tighter can create a custom arch specific to an individual patient's anatomy. When the elongated gripping members 104 and 106 are no longer manipulated, the soft tissue retracting members 224 and 244 are urged back to their normal angular positions relative to each other (i.e., in the substantially constricted or contracted state as represented by the solid arrows) by a biasing force of the biasing element 102. Protrusions or tabs 230 and 250 act as stops during non-use of the mandibular retractor 12.

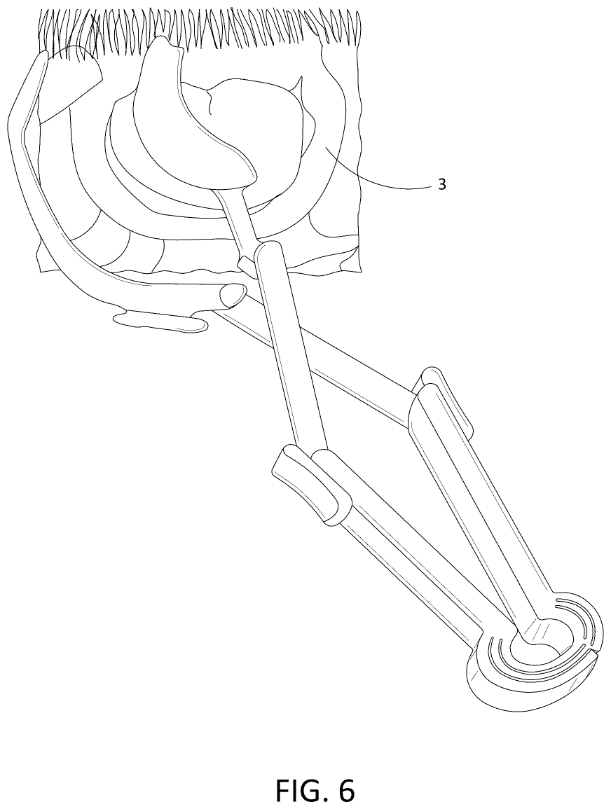

The characteristics of the mandibular retractor 12, as described above, provides the device with many advantages, making it highly suitable to retract soft tissue as necessary and to provide a clear view of the area of interest, while ensuring comfort to the patient during any procedure, as may be understood from FIG. 6.

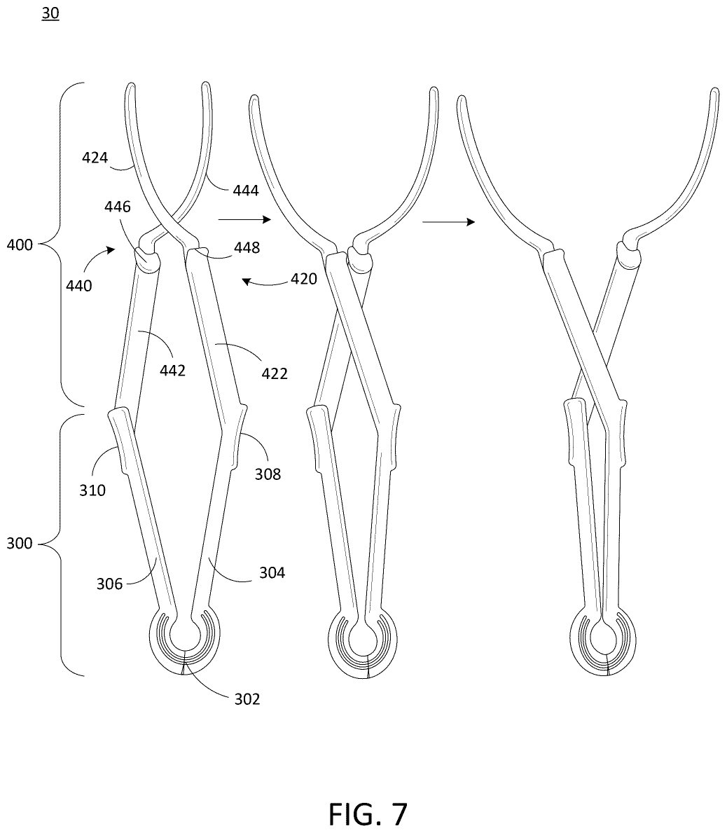

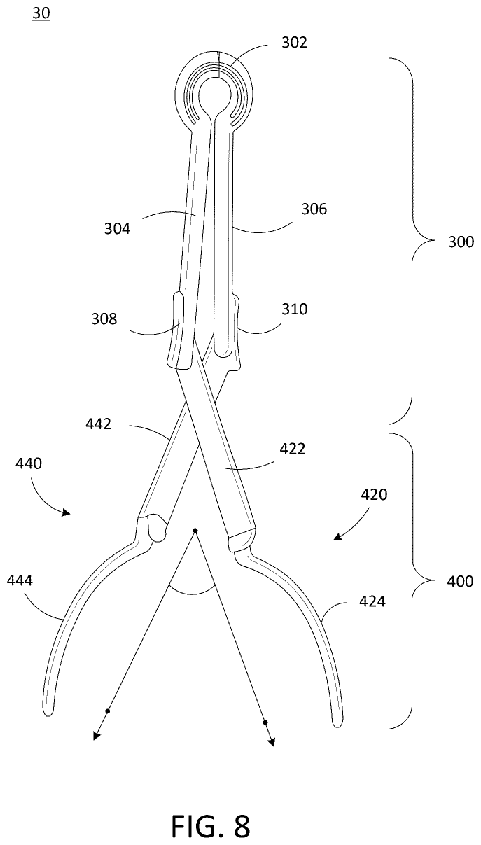

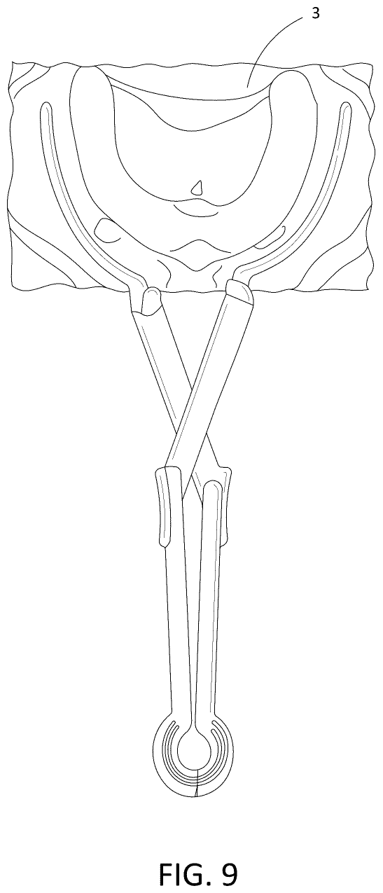

Turning to FIGS. 7-9, these figures depict another embodiment according to the present invention in the form of a maxillary retractor 30. The retractor 10 may be a maxillary retractor 30 may comprise a controlling handle portion 300 and a soft tissue retracting portion 400. The maxillary retractor 30, like the mandibular retractor 12, may be in the form of a single unitary structure or may comprise multiple attached (i.e., connected, fastened, fused, or bonded) components that may be assembled together according to conventional techniques.

The controlling handle portion 300 of maxillary retractor 30 may include a biasing element 302 (e.g., a generally arch- or C-shaped spring element or other suitable biasing element identical or similar to those described for retractor 10) and two elongated gripping members 304 and 306. The two elongated gripping members 304 and 306 may be provided with finger pads or rests 308 and 310, respectively, for ergonomic operation—i.e., user comfort and easy manipulation.

The soft tissue retracting portion 400 may extend from or may be attached (i.e., connected, fastened, fused, or bonded) to the elongated gripping members 304 and 306. The soft tissue retracting portion 400 may comprise two arms 420 and 440, which may extend from elongated gripping members 304 and 306, respectively. Arm 420 may include elongated extension member 422 (proximal to elongated gripping member 304) and soft tissue retracting member 424 (distal to elongated gripping member 304), and it may be contoured in shape to conform to the patient's oral anatomy. Similarly, arm 440 may include elongated extension member 442 (proximal to elongated gripping member 306) and soft tissue retracting member 444 (distal to elongated gripping member 306), and it may be contoured in shape to conform perfectly to the patient's oral anatomy. The elongated extension members 422 and 442 may be configured to provide adequate placement into the vestibule of a patient's mouth.

Additionally, protrusions or tabs 446 and 448 may be provided on either the elongated extension members 442 and 422, respectively, or on soft tissue retracting sections 444 and 424, respectively, to act as stops.

As best seen in FIG. 7, the biasing element 302 may be configured to ensure that the soft tissue retracting members 424 and 444 are substantially constricted or contracted in relation to each other when the maxillary retractor 30 is not in use and expanded when the controlling handle portion 300 is manipulated by pressing or squeezing finger pads or rests 308 and 310 after placement into the patient's oral cavity 3. Unlike mandibular retractor 12, the elongated extension members 422 and 442 of maxillary retractor 30 intersect in an overlaid relation when pressing or squeezing force is applied on finger rests or tabs 308 and 310 but not in the non-use position (FIG. 7, left). In the non-use position, only portions of the soft tissue retracting members 424 and 444 intersect in an overlaid configuration.

In another aspect, the maxillary retractor 30 may optionally be provided with hamular notch extensions for proper retraction. Specifically, a hamular notch is an anatomical feature of the maxilla, which may provide additional extension with further curvature to utilize the specific anatomy of the hamular notch to provide retraction in this area which could be helpful in scanning some individuals.

Again, it should be appreciated that any part(s) of the maxillary retractor 30 may be constructed to be unitary or monolithic with other part(s) of the maxillary retractor 30. The maxillary retractor 30 may be made from various suitable materials including, but not limited to, one or more of metallic materials, polymeric materials (e.g., PLA), reinforced fiber materials (carbon fiber-containing materials), composites, and combinations thereof. The materials of construction for maxillary retractor 30 may be selected to form a device that is either disposable (in whole or part) or completely reusable (with autoclaving capability).

In all embodiments, the expandable angular range of the retractors may be configured for a given application. Typically, an expandable angular range of about 10 to about 110 degrees may be sufficient, particularly for the maxillary retractor 30.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of coverage of the invention should be determined by the appended claims and their legal equivalents.

What is claimed is:

1. A retractor comprising:
a controlling handle portion comprising a biasing element, a first elongated gripping member, and a second elongated gripping member, wherein each elongated gripping member extends from or is attached to the biasing element; and
a soft tissue retracting portion comprising a first arm and a second arm, wherein each arm has a proximal section comprising elongated extension members that extend from or is attached to one of the elongated gripping members of the controlling handle portion and a distal section comprising a soft tissue retracting member that is configured to contact and retract tissue;
wherein the controlling handle portion and the soft tissue retracting portion are configured such that the soft tissue retracting members are positioned in a substantially contracted state in relation to each other by a biasing force of the biasing element when the retractor is not in use but expandable in angular relation to each other when the first and second elongated gripping members are pressed towards each other.

2. The retractor according to claim 1, wherein the elongated extension members intersect in an overlaid, scissor configuration.

3. The retractor according to claim 1, wherein the biasing element has a curved shape.

4. The retractor according to claim 2, wherein the biasing element has a generally arch- or C-shape.

5. The retractor according to claim 1, wherein the biasing element is in a form of a spring.

6. The retractor according to claim 1, further comprising finger pads or rests on both elongated gripping members.

7. The retractor according to claim 1, wherein the elongated extension members are provided with a slide system or ratchet system for locking the elongated gripping members in place.

8. The retractor according to claim 1, wherein the elongated extension members are provided with notches and recesses for locking the elongated gripping members at a selected angle.

9. The retractor according to claim 1, wherein the elongated extension members or the soft tissue retracting members are provided with protrusions or tabs to act as stops against the biasing force exerted by the biasing element.

10. The retractor according to claim 1, wherein the soft tissue retracting members has a range of expandable angular displacement of about 10 to about 110 degrees.

11. The retractor according to claim 1, in a form of a unitary structure.

12. The retractor according to claim 1, wherein retractor is manufactured from metal, polymeric material(s), fiber-reinforced material, or combinations thereof.

13. The retractor according to claim 11, wherein the retractor is autoclavable.

14. The retractor according to claim 1, wherein at least the soft tissue retracting member is disposable.

15. A mandibular retractor comprising:
a controlling handle portion comprising a curved biasing element, a first elongated gripping member, and a second elongated gripping member, wherein each elongated gripping member extends from or is attached to the curved biasing element; and
a soft tissue retracting portion comprising a first arm and a second arm, wherein each arm has a proximal section comprising elongated extension members that extend from or is attached to one of the elongated gripping members of the controlling handle portion and a distal section comprising a soft tissue retracting member that is configured to contact and retract tissue;
wherein the controlling handle portion and the soft tissue retracting portion are configured such that the soft tissue retracting members are positioned in a substantially contracted state in relation to each other by a biasing force of the biasing element when the retractor is not in use but expandable in angular relation to each other when the first and second elongated gripping members are pressed towards each other.

16. The mandibular retractor according to claim 15, wherein one of the soft tissue retracting member includes a buccal shelf contour, a retromolar pad contour, and a labial vestibule extension.

17. The mandibular retractor according to claim 15, wherein one of the soft tissue retracting member includes a glossal hood, a retromylohyoid fossas extension, and a lingual vestibule extension.

18. A maxillary retractor comprising:
a controlling handle portion comprising a curved biasing element, a first elongated gripping member, and a second elongated gripping member, wherein each elongated gripping member extends from or is attached to the curved biasing element; and
a soft tissue retracting portion comprising a first arm and a second arm, wherein each arm has a proximal section comprising elongated extension members that extend from or is attached to one of the elongated gripping members of the controlling handle portion and a distal section comprising a soft tissue retracting member that is configured to contact and retract tissue;
wherein the controlling handle portion and the soft tissue retracting portion are configured such that the soft tissue retracting members are positioned in a substantially contracted state in relation to each other by a biasing force of the biasing element when the retractor is not in use but expandable in angular relation to each other when the first and second elongated gripping members are pressed towards each other.

19. The maxillary retractor according to claim 18, wherein the soft tissue retracting members are shaped to conform anatomically.

20. The maxillary retractor according to claim 18, wherein the elongated extension members intersect only when the retractor is expanded.
